(19) United States
(12) Patent Application Publication
(10) Pub. No.: US 2008/0004913 A1
(43) Pub. Date: Jan. 3, 2008

(54) Pharmaceutical safety terminal

(76) Inventor: Garrett Stewart Sayre, Yorktown, VA (US)

Correspondence Address: Garrett S. Sayre, 1900 I Piccadilly Loop, Yorktown, VA, 23692, US

(21) Appl. No.: 11/716,786

(22) Filed: Mar. 12, 2007

Related U.S. Application Data
(60) Provisional application No. 60/817,235, filed on Jun. 29, 2006.

(51) Int. Cl. A61B 5/00

(52) U.S. Cl. 705/3

(57) ABSTRACT

The Pharmaceutical Safety Terminal, PST, uses a computer download of the physician's desk reference, PDR, or similar data as one source of reference input. The other primary source of data input is the patient's medical profile. The PST automatically calculates the perceived safety of each medication with the risk factor in the patient's profile. On the side of this approximately 3 inch by 3½ inch device is a green, red, and yellow light. If the red light shows the doctor must realize that the medication is strongly counter indicated. Yellow means use caution and green means there are no exceptional warning signs. The PST slides on the physician's belt so he will carry it at all times. The PST also gives a graphic read out of the possible side effects of each drug typed into the patient's profile. So the doctor can read the specifics of each drug interaction before it is administered to the patient. At the end of the work shift the PST is then plugged into a central computer for any downloads of new data relevant to the drugs or patient's status for the highest possible expectation of patient safety. Critical information can be automatically emailed from the source computer network. If there is a specifically critical new warning, the PST will activate the red light during download for the physician to pay specific attention to this download when he picks up the device at the beginning of his shift. Every medication given to the patient even by another physician must be entered or downloaded in the PST to avoid adverse synergistic effects. Another function of the PST will be to enlist potential antidotes for the wrongly administered drug and what danger signs are for the wrongly administered drug and display phone numbers of the world's top experts in countering the effects for possible personal consultation. Since doctors kill hundreds of thousands of patients by giving them the wrong medication this will sharply reduce deaths and malpractice suits.

### PATENT APPLICATION FOR LIFESAVING MEDICAL DEVICE, THE PHARMACEUTICAL SAFETY TERMINAL

This application takes advantage of the previously applied for provisional patent: 60/817,235

Non Provisional application Ser. No. 11/716,786

Filing Date Jun. 29, 2006

Inventor: Garrett S. Sayre

Residence: 1900 I Piccadilly Loop, Yorktown, Va. 23692 Phone: 757-236-0459

Title: Pharmaceutical Safety Terminal

### FIELD OF INVENTION

This invention relates to saving peoples lives by implementing the highest possible safety precautions using a computerized process to avoid physician initiated errors when administering drugs

### BACKGROUND OF THE INVENTION

Medical practitioners kill or injure a million and a half patients a year in the United States by incorrectly administering drugs. The pain and suffering this causes is incalculable. It also dramatically increases malpractice suits. For the benefit of the patient, parents, family and friends, the doctor and the hospital a safer method must be used. The Pharmaceutical Safety Terminal is such a device. The Pharmaceutical Safety Terminal (PST), utilizes a computer process that calculates the safety of a drug in relation to a patients medical profile.

### SUMMARY OF INVENTION

The objective of this invention is to save lives and alleviate human suffering. The objective is a Computerized Process by which drug information similar to that of the Physicians Desk Reference is contrasted with a patient's medical profile, including gender, age, height, weight to determine the safety of a certain prescription and indicate its effects.
The objective of this inventions is a process by which the patient's profile can be entered and the computer listing certain medication options.
The objective of this process is to avoid physician's error when prescribing medication that leads to the death or injury of patients.
The objective of this process is to avoid negative synergistic effects when combining medications.
The objective of this process is to indicate danger signs for a wrongly administered drug.
The objective of this process is to give contact information for experts and counter effects, antidotes for wrongfully administered drugs and harmful or deadly drug interactions.
The objective of this process is to indicate allergy alerts for each patient.
The objective of this process is to produce a research history for any drug being prescribed.
Another objective is to produce a label (or other document) with any appropriate medical information.
Another objective is to include an automated e-mail system, which would update patient's information from other automated systems.
Another objective is to include a manually incorporated email system to send off an authorization list individuals the appropriate patient information.
Another objective is a drug/patient name alignment system by pressing the drug button and spelling as much of the drug as necessary then pressing the patient button and spelling as much of the patient's name as necessary then pressing O.K., for results to readout.
Another objective is to include a standard download/upload system when attached to the main computer medical database.
Another objective is to adapt this program for veterinary use.
Another objective is to adapt this program for use by emergency service officers.
Another objective is for the program to display possible antidotes and contact information of the world's top experts for immediate help in reversing the effects of wrongly administered drugs and harmful or deadly drug interaction.
Another objective is to indicate danger signs for a wrongly administered drug or drugs.
Another objective of the program is to include clear indicating signals to determine the threat level of the drug interaction. These indications include Green (considered reasonably safe), Yellow (physician must weigh to options by the totality of the circumstances, including getting a research history on the drug), Red (harmful or deadly). An alpha numeric read out in the nature of written and mathematical information (such as a simple sentence combined with a risk percentage). Tones such as a happy tone for safe, a suspenseful tone for uncertain, an ominous tone for harmful or deadly.
Another objective is a scrollbar system to quickly match up patient name with the name of the drug and press O.K. for input.

What is claimed is:

1. A computer program that stores all patient medication information and indicates its effects in relation to the complete patient profile of the individual person.

2. A computer device that is approximately three inches by three and a half inches in size on which the software in claim 1 is housed.

3. The computer program of claim 1, wherein said program indicates overdose if medication is over-prescribed based on patient's weight, general health, age and sex.

4. The computer program of claim 1, wherein said program indicates potential dangerous interactions based on patient medical history.

5. The computer program of claim 1, wherein said program indicates allergy alerts for each patient.

6. The computer program of claim 1, wherein said program produces a research history for any drug being prescribed.

7. The computer program of claim 1, wherein said program produces a label (or other document) with any appropriate medical information.

8. The appearance of the device in claim 2 consists of flashing green, yellow and red lights to indicate threat level or an alpha/numeric or sonic designation for the same purpose.

9. The function of the program in claim 1 being to activate flashing green, yellow and red lights to indicate threat level or an alpha/numeric or sonic designation for the same purpose.

10. The apparatus of the software in claim 1, consisting of an automatic e-mail system, which updates and receives patient information on/from other authorized systems.

11. The apparatus of claim 1, consisting of a manually incorporated e-mail system to send off-list individuals the appropriate patient information.

12. The apparatus of claim 1, consisting of a standard download/upload system when attached to the main computer medical database.

13. The adaptability of the software in claim 1, wherein said program could be adapted for use by emergency service officers.

14. The adaptability of the software in claim 1, where in said program could be adapted for use by veterinarians.

15. The method of the computer program in claim 1, where in the program uses a reverse process by listing possible medication options based on a patient's medical profile.

16. The computer program of claim 1, wherein said program displays possible antidotes and contact information of the world's top experts for immediate help in reversing the effects of wrongly administered drugs and harmful or deadly drug interactions.

17. The computer program of claim 1, wherein said program indicates danger signs for a wrongly administered drug or drugs.

18. The method of the device in claim 2, wherein said device includes a scrollbar for patient identification (names) and drug identification (names) that can be scrolled to and matched up by the physician saving input time.

19. The method of the program in claim 1, wherein said program uses a drug/patient name alignment system by pressing the drug button on the device spelling as much of the drug name as necessary then pressing the patient button and spelling as much of the patient's name as necessary then pressing O.K. for the results readout.

20. The method of the device in claim 2, wherein said device uses a drug/patient name alignment system by pressing the drug button on the device spelling as much of the drug name as necessary then pressing the patient button and spelling as much of the patient's name as necessary then pressing O.K. for the results readout.
